(19) United States
(12) Patent Application Publication
(10) Pub. No.: US 2012/0129803 A1
(43) Pub. Date: May 24, 2012

(54) COMPOSITION CONTAINING CHITOOLIGOSACCHARIDE FOR RECOVERING FROM FATIGUE

(75) Inventors: Eui Seok Shin, Yongin-si (KR); Si Young Cho, Seoul (KR); Pil Joon Park, Yongin-si (KR); Jong Hee Sohn, Yongin-si (KR); Min Jung Song, Anyang-shi (KR); Ji Hea Lee, Yongin-si (KR); Dae Bang Seo, Yongin-si (KR); Sang Jun Lee, Sungnam-si (KR)

(73) Assignee: Amorepacific Corporation, Seoul (KR)

(21) Appl. No.: 13/387,495

(22) PCT Filed: Jul. 28, 2010
(86) PCT No.: PCT/KR10/04947

§ 371 Date: Jan. 27, 2012

(30) Foreign Application Priority Data
Jul. 28, 2009 (KR) 10-2009-0068784

(51) Int. Cl. A61K 31/722; A61P 25/00; C08B 37/08

(52) U.S. Cl. 514/55; 536/20

(57) ABSTRACT

Disclosed is an anti-fatigue composition containing chitooligosaccharide as an active ingredient. The composition exhibits a fatigue recovery effect, and thus can be widely used in the health food or medical field.

### TECHNICAL FIELD

The present invention relates to a composition for recovery from fatigue containing chitooligosaccharide as an active ingredient.

### BACKGROUND ART

Generally, fatigue means a state in which motor or cognitive ability decreases even though there is no abnormality in muscular tissue. Prolonged fatigue adversely affects the quality of life, and particularly, accumulated fatigue affects not only the muscular system, but also the nervous system. Fatigue can cause aging, endocrine and metabolic diseases, cardiac diseases, respiratory diseases, etc., and can be caused by lifestyle habits such as overwork or stress.

Fatigue significantly reduces the quality of life and the efficiency of work and can lead to diseases in severe cases. For example, continuous fatigue can lead to chronic fatigue syndromes. Rest is known to be most effective in removing fatigue. Particularly, it is known that recovery from chronic fatigue syndrome is not easy even by rest.

These days, many persons suffer from chronic fatigue due to overwork and stress, but an effective drug for removing fatigue is not known. Although drugs having an awakening effect, such as caffeine, are used as drugs for recovery from fatigue, they are not effective in reducing blood fatigue material.

For recovery from fatigue, energy production in vivo is required. Intracellular organelles that are involved in energy production at the cell level are mitochondria. Mitochondria that are intracellular organelles are present in most eukaryotic cells and themselves have mitochondrial DNA (mtDNA), which is different from nuclear DNA. The major function of mitochondria is to produce ATP (adenosine triphosphate) that is an intracellular energy source. ATP is produced in the electron transport system using NADH and FADH₂ which are produced by the TCA cycle in the matrix. The produced ATP is used to derive energy for biosynthesis and various metabolic activities. Also, mitochondria function to store calcium ions playing an important role in intracellular signaling and to supply the stored calcium ions to the cytoplasm as needed. In addition, mitochondria are known to regulate apoptosis, proliferation and metabolism.

Particularly, unlike cellular nuclear DNA, mitochondrial DNA (mtDNA) is easily damaged, because it has no repair mechanism and does not have a histone protein functioning to protect DNA. This damage to mitochondrial DNA (mtDNA) is known to have a close connection with the onset of mitochondrial disease. Also, damage to mitochondrial DNA (mtDNA) leads to a decrease in mitochondrial functions, thus reducing the synthesis of ATP, an energy source required for cellular activity, and causing various diseases.

Chitosan is used in a wide range of applications, including coagulants for wastewater treatment, heavy metal adsorbents, functional food products, ion exchangers, and medical products. Such functional properties are known to be greatly influenced by the molecular weight and deacetylation of chitosan. Methods that are generally used to control the molecular weight of chitosan include enzymatic and chemical degradation methods. However, the chemical degradation method causes environmental problems by wastewater treatment, because it uses excessive amounts of strong acids or bases.

In recent years, it has been known that chitin, chitosan and derivatives thereof adsorb and excrete harmful cholesterols accumulated in the body, inhibit the proliferation of cancer cells, adsorb chloride ions causing an increase in blood pressure, inhibit the intestinal absorption of chloride ions, excrete chloride ions from the body, promote the proliferation of intestinal beneficial bacteria and activate cells. In addition, they regulate blood glucose levels, improve liver functions, and remove heavy metals and contaminants from the body. Due to such various physiological activities, these materials have been much studied as promising materials having high added values in the biomedical field.

As is known from previous studies, chitooligosaccharide that is a low-molecular-weight polysaccharide obtained by hydrolyzing chitosan by acids or enzymes shows higher in vivo absorption than chitosan and has immune enhancing effects, antioxidant effects and inhibitory effects on the growth of cancer cells. Also, chitooligosaccharide is known to have the effect of inhibiting liver damage induced by carbon tetrachloride.

### DISCLOSURE

### Technical Problem

An object of one embodiment of the present invention is to provide a composition having a fatigue recovery effect.

### Technical Solution

A composition according to the present invention for achieving this object contains chitooligosaccharide lactate as an active ingredient.

### Advantageous Effects

The composition according to the present invention shows the effects of preventing fatigue accumulation and recovering from fatigue. Thus, the composition of the present invention can be widely used in the health food or medical field.

### DESCRIPTION OF DRAWINGS

FIG. 1 is a graphic diagram showing the duration of immobility of mice, measured after chitooligosaccharide lactate according to one embodiment of the present invention was added to the mice.

FIG. 2 is a graphic diagram showing the amount of mitochondria in the platelet of mice, measured after chitooligosaccharide lactate according to one embodiment of the present invention was added to the mice.

### BEST MODE

An anti-fatigue composition according to one embodiment of the present invention contains chitooligosaccharide lactate as an active ingredient.

The molecular weight of the chitooligosaccharide in the composition may be 700-9,000.

The chitooligosaccharide may activate mitochondria and increase the copy number of mitochondria.

This anti-fatigue composition may be a health food composition or a pharmaceutical composition.

The content of the chitooligosaccharide in the health food composition or the pharmaceutical composition may be, for example, about 10-90 wt % based on the total weight of the composition.

### MODE FOR INVENTION

A composition according to one embodiment of the present invention contains chitooligosaccharide as an active ingredient and may be used for recovery from fatigue.

As used herein, the term “chitooligosaccharide” means a low-molecular-weight polysaccharide resulting from the hydrolysis of chitosan. The term “low molecular weight” is a term for a collection of materials having low molecular weights, for example, less than 10,000, particularly 9,000 or less. More specifically, the molecular weight of chitooligosaccharide in the composition according to the present invention may be 700-9,000.

A method for preparing chitooligosaccharide is not specifically limited. For example, chitooligosaccharide may be prepared by crushing, desalting, deproteinizing and purifying crab or shrimp shells to obtain chitin, deacetylating the chitin to prepare chitosan, and chemically or enzymatically degrading the prepared chitosan. Particularly, the chitooligosaccharide may be prepared by enzymatic degradation.

In the case in which chitooligosaccharide is prepared by enzymatic degradation of chitosan, the enzyme that is used in the enzymatic degradation is not specifically limited and may be, for example, cellulase.

A method of preparing chitooligosaccharide by enzymatic degradation of chitosan is not specifically limited. For example, purified water is added to chitosan, lactic acid is added thereto in an amount of 2-3%, and the mixture is stirred at a temperature of 40˜60° C., thus preparing a lactic acid-containing chitosan dispersion having a solid content of 5-10%. The chitosan dispersion is adjusted to a pH of 4-6, and a solution of cellulase (chitosan degrading enzyme) in purified water is added thereto. Then, the mixture is subjected to hydrolysis at 40˜60° C. for 14-20 hours, after which it is heat-treated at 80° C. for 30 minutes to deactivate the enzyme, and filtered and dried, thereby preparing chitooligosaccharide.

In one embodiment, the molecular weight of chitooligosaccharide may be in the range of 700 to 9,000, and a chitooligosaccharide having a molecular weight within this range can exhibit an excellent effect of activating mitochondria. The molecular weight of chitooligosaccharide can vary depending on the amount of cellulase added during the preparation thereof. For example, when the cellulase enzyme is added in an amount of 10% of chitosan, a chitooligosaccharide having a molecular weight of 1,000 or less can be obtained, and when cellulase is added in an amount of 6%, a chitooligosaccharide having a molecular weight of 1,500-2,000 can be obtained. When cellulase is added in an amount of 3%, a chitooligosaccharide having a molecular weight of 7,000-10,000 can be obtained. Thus, the molecular weight of chitooligosaccharide prepared by enzymatic degradation of chitosan may be in the range of 700-9,000.

In one embodiment, the composition contains chitooligosaccharide as an active ingredient and can show a fatigue recovery effect by activating mitochondria. The chitooligosaccharide may be in the form of various salts. For example, the chitooligosaccharide may be a chitooligosaccharide lactate or hydrochloride. Particularly, the chitooligosaccharide may be a chitooligosaccharide lactate. In the Examples of the present invention, it was found that, in a test group administered with a chitooligosaccharide lactate having an average molecular weight of 1155 among various salts of chitooligosaccharide, mitochondria were increased or activated, thereby exhibiting a fatigue recovery effect. It was shown that the chitooligosaccharide lactate had the effect of significantly increasing the mitochondrial copy number.

In one embodiment, the composition according to the present invention may contain chitooligosaccharide lactate as an active ingredient and may be a composition showing a fatigue recovery effect or a composition for treating chronic fatigue syndrome.

The composition may be a health food composition or a pharmaceutical composition, but is not specifically limited thereto.

The health food composition may be formulated in various forms, including powders, granules, tablets, capsules and drinks.

The health food composition may, if necessary, contain one or a mixture of two or more selected from among, for example, fruit juice (including concentrated fruit juice and powdered fruit juice) such as grapefruit juice, apple juice, orange juice, lemon juice, pineapple juice, banana juice, pear juice, etc.; vitamins (water-soluble and fat-soluble vitamins) such as retinol palmitate, riboflavin, pyridoxine, cyanocobalamin, sodium ascorbate, nicotinamide, calcium pantothenate, folic acid, biotin, cholecalciferol, choline bitartrate, tocopherol, p-carotene, etc.; flavors such as lemon flavor, orange flavor, strawberry flavor, grapefruit flavor, vanilla essence, etc.; amino acids, nucleic acids and salts thereof such as glutamic acid, sodium glutamate, glycine, alanine, aspartic acid, sodium aspartate, inosinic acid, etc.; plant fibers such as polydextrose, pectin, xanthan gum, glucomannan, alginic acid, etc.; and minerals such as sodium chloride, sodium acetate, magnesium sulfate, potassium chloride, magnesium chloride, magnesium carbonate, calcium chloride, dipotassium phosphate, monosodium phosphate, calcium glycerophosphate, sodium ferrous citrate, ammonium ferric citrate, ferric citrate, manganese sulfate, copper sulfate, sodium iodide, potassium sorbate, zinc, manganese, copper, iodine, cobalt, etc.

The pharmaceutical composition may further comprise a pharmaceutical adjuvant such as a preservative, a stabilizer, a hydrating agent, an emulsifying accelerator, a salt for control of osmotic pressure, a buffer, etc. and other therapeutically useful substances. The pharmaceutical composition may be formulated into various forms for oral or parenteral administration.

The formulation for oral administration may include, for example, tablet, pill, hard or soft capsule, liquid, suspension, emulsion, syrup, granule, or the like. These formulations may include, in addition to the active ingredient, a diluent (e.g., lactose, dextrose, sucrose, mannitol, sorbitol, cellulose and glycine), a lubricant (e.g., silica, talc, stearic acid or its magnesium or calcium salt, and polyethylene glycol). The tablet may further include a binder such as magnesium aluminum silicate, starch paste, gelatin, tragacanth, methyl cellulose, sodium carboxymethyl cellulose, and polyvinylpyrrolidine. As occasion demands, it may further include a pharmaceutical additive such as a disintegrant, e.g., starch, agar, alginic acid or its sodium salt, an absorbent, a colorant, a flavoring agent, a sweetener, or the like. The tablet may be prepared according to conventional mixing, granulation or coating methods. Typical formulations for parenteral administration include isotonic aqueous solutions or suspensions for injection.

The dose of the active ingredient may be easily determined by those skilled in the art. A daily dose of the active ingredient may vary depending on various factors, including the progression and stage of the disease, the age and physical condition of the patient, the presence or absence of complications, and so forth. In general, the composition having the aforesaid weight ratio may be administered once or twice a day, in a total daily amount of 1-500 mg/kg, particularly 30-200 mg/kg, for adults. However, the described administration dose does not limit the scope of the present invention in any way.

The content of chitooligosaccharide in the composition may be, but is not specifically limited to, 10-90 wt %, and preferably 20-50 wt %, based on the total weight of the composition. Specifically, a tablet or a soft capsule may include the active ingredient in an amount of 10-60 wt %, and a hard capsule may include the active ingredient in an amount of 10-90%. The present invention may provide a health food composition or a pharmaceutical composition, which contains chitooligosaccharide in an amount of 10-90 wt %.

Hereinafter, the present invention will be described in further detail with reference to examples. It is to be understood, however, that these examples are for illustrative purposes only and are not intended to limit the scope of the present invention.

In the present invention, the effect of chitooligosaccharide lactate on mitochondrial production and the anti-fatigue effect thereof were analyzed in animal studies. Specifically, the amount of mitochondria in the blood of mice administered with chitooligosaccharide lactate was measured and the anti-fatigue effect of chitooligosaccharide lactate was measured in a forced swimming test.

### TEST EXAMPLE 1

### Test Animals and Test Method

3-week old hairless mice were purchased and housed in groups of 10 mice per cage. In order to minimize the effect of the hair of mice in a forced swimming test, hairless mice were used. The animals were allowed feed and water ad libitum and kept at a temperature of 22±1° C. and a humidity of 60±5% under a 12-hr light/12-hr dark cycle. The test animals were divided into a fatigue group and a non-fatigue group, and the fatigue group was further divided into a hydrochloride-administered group, a lactate-administered group and a non-administered group.

TABLE 1

| | | | Number of test |
|---|---|---|---|
| Test group | | Test material | animals |
| Non-fatigue group | | — | 10 |
| Fatigue | Non-administered | — | 10 |
| group | group | | |
| | Test group 1 | chitooligosaccharide | 10 |
| | | lactate | |
| | Test group 2 | Chitooligosaccharide | 10 |
| | | hydrochloride | |

The test group was administered with a test material at a dose of 500 mg/kg for 2 weeks. After 2 weeks of administration. Fatigue in the fatigue group was induced by fixing the mouse cage onto a shake and shaking the mouse cage for 24 hours. The mice shaken for 24 hours and the mice not subjected to fatigue were subjected to a forced swimming test.

### TEST EXAMPLE 2

### Forced Swimming Test for Measuring Anti-Fatigue Effect in Mice

A forced swimming test (FST) is an animal test method that is frequently used to determine the degree of depression and has been widely used as a preclinical test method for verifying the effect of an antidepressant. Also, FST has been used to verify the effects of a specific substance on anti-fatigue and endurance (Koo H N et al., Biol Pharm Bull, 27, 117-119, 2004; Shin H Y et al, Biol Pharm Bull, 27, 1521-1526, 2004).

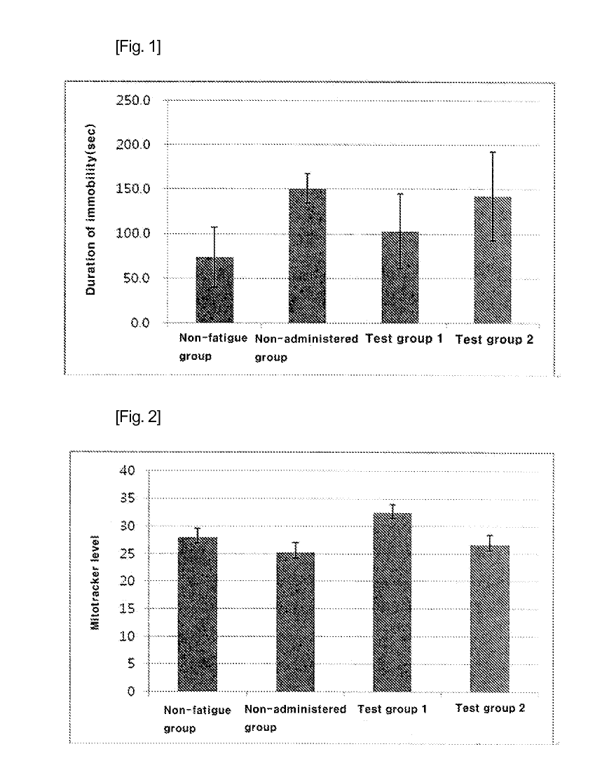

In FST, the duration of immobility of mice was recorded for 6 minutes. Two opaque glass cylinders (height: 25 cm; diameter: 10 cm) were filled with water up to a height of 10 cm and two mice were simultaneously allowed to enter the water and stabilized for 2 minutes. After stabilization, the duration of immobility of the mice was recorded during the last 4 minutes. A mouse was considered immobile when floating with the head above the surface of water without active movement. The results of the test are shown in FIG. 1.

As shown in FIG. 1, the duration of immobility was significantly reduced in the chitooligosaccharide lactate-administered group compared to the non-administered group and the group that was subjected to fatigue after administering hydrochloride. This suggests that chitooligosaccharide shows a fatigue recovery effect and particularly that chitooligosaccharide lactate has an excellent anti-fatigue effect compared to chitooligosaccharide hydrochloride.

### TEST EXAMPLE 3

### Separation of Platelet from Mouse Blood

After the forced swimming test, the mouse blood was collected and treated with an anticoagulant. After treatment with the anticoagulant, the blood was centrifuged at a speed of 150×g for 10 minutes to separate plasma containing concentrated platelet. To separate platelet, the plasma containing concentrated platelet was centrifuged at a speed of 300×g for 10 minutes. The precipitated platelet was dissolved in a Tyrod buffer (137 mM sodium chloride, 12 mM NaHCO₃, 5.5 mM glucose, 2 mM KCl, 1 mM MgCl₂, 0.3 mM Na₂HPO₄, pH 7.4) and analyzed.

### TEST EXAMPLE 4

### Measurement of Copy Level of Mitochondria in Platelet

To measure the amount of mitochondria in the platelet, 50 μl of the platelet was stained with 25 nM of mitotracker Red (Molecular Probes, Invitrogen) at 37° C. for 30 minutes and analyzed by flow cytometry. The results of the analysis are shown in FIG. 2.

As can be seen in FIG. 2, the amount of mitochondria significantly increased in the group administered with chitooligosaccharide lactate or hydrochloride compared to the fatigue group. Particularly, the amount of mitochondria in the group administered with chitooligosaccharide lactate remarkably increased.

This suggests that chitooligosaccharide shows the effect of increasing the amount of mitochondria and particularly that chitooligosaccharide lactate shows an excellent effect of increasing the amount of mitochondria compared to chitooligosaccharide hydrochloride.

Formulation examples of the composition will now be described, but are for illustrative purposes only and are not intended to limit the scope of the present invention.

### FORMULATION EXAMPLE 1

### Preparation of Soft Capsule

80 mg of chitooligosaccharide, 9 mg of vitamin E, 9 mg of vitamin C, 2 mg of palm oil, 8 mg of hydrogenated vegetable oil, 4 mg of beeswax and 9 mg of lecithin were mixed with each other according to a conventional method to prepare a solution for filling into soft capsules. 400 mg of the solution was filled into each soft capsule, thus preparing a soft capsule formulation. Meanwhile, a soft capsule sheet was prepared using 66 parts by weight of gelatin, 24 parts by weight of glycerin and 10 parts by weight of sorbitol, and then filled with the above filling solution to prepare a soft capsule formulation containing 400 mg of the composition according to the present invention.

### FORMULATION EXAMPLE 2

### Preparation of Tablet

80 mg of chitooligosaccharide, 9 mg of vitamin E, 98 mg of vitamin C, 200 mg of galactooligosaccharide, 60 mg of lactose and 140 mg of maltose were mixed with each other, granulated using a fluidized-bed dryer, and then 6 mg of sugar ester was added thereto. 504 mg of the resulting composition was compressed into a tablet according to a conventional method.

### FORMULATION EXAMPLE 3

### Preparation of Drink

80 mg of chitooligosaccharide, 9 mg of vitamin E, 9 mg of vitamin C, 10 g of glucose, 0.6 g of citric acid and 25 g of liquid oligosaccharide were mixed with each other. After adding 300 ml of purified water thereto, 200 ml of the mixture was filled in each bottle which was then sterilized at 130° C. for 4-5 seconds, thereby preparing a drink.

### FORMULATION EXAMPLE 4

### Preparation of Granule

80 mg of chitooligosaccharide, 9 mg of vitamin E, 9 mg of vitamin C, 250 mg of anhydrous crystalline glucose and 550 mg of starch were mixed with each other, granulated using a fluidized-bed granulator, and then filled in a pouch, thereby preparing a granule formulation.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

### INDUSTRIAL APPLICABILITY

The composition according to the present invention shows the effects of preventing fatigue accumulation and recovering from fatigue. Thus, the composition of the present invention can be widely used in the health food or medical field.

1. An anti-fatigue composition containing chitooligosaccharide lactate as an active ingredient.

2. The anti-fatigue composition of claim 1, wherein the molecular weight of the chitooligosaccharide is 700-9,000.

3. The anti-fatigue composition of claim 1, wherein the chitooligosaccharide functions to activate mitochondria.

4. The anti-fatigue composition of claim 1, wherein the chitooligosaccharide functions to increase the copy number of mitochondria.

5. The anti-fatigue composition of claim 1, wherein the composition is a health food composition or a pharmaceutical composition.

6. The anti-fatigue composition of claim 5, wherein the content of the chitooligosaccharide in the composition is 10-90 wt % based on the total weight of the composition.

7. A method of combating fatigue comprising administering to a subject in need thereof an effective amount of chitooligosaccharide lactate.

8. The method of claim 7, wherein the molecular weight of the chitooligosaccharide is 700-9,000.

9. The method of claim 7, wherein the fatigue is chronic fatigue.

10. The method of claim 7, wherein the amount of chitooligosaccharide administered is 1 to 500 mg/kg.

11. The method of claim 10, wherein the amount administered is 30 to 200 mg/kg.
